(19) United States
(12) Patent Application Publication
(10) Pub. No.: US 2007/0116767 A1
(43) Pub. Date: May 24, 2007

(54) Chitosan-microparticles for ifn gene delivery

(76) Inventor: Shyam S. Mohapatra, Tampa, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/544,145

(22) PCT Filed: Feb. 13, 2004
(86) PCT No.: PCT/US04/04262

§ 371 Date: Dec. 22, 2006

Related U.S. Application Data
(60) Provisional application No. 60/319,946, filed on Feb. 14, 2003.
(60) Provisional application No. 60/319,956, filed on Feb. 19, 2003.

(51) Int. Cl. A61K 48/00; A61K 9/14

(52) U.S. Cl. 424/489; 514/44; 514/55; 977/906

(57) ABSTRACT

The present invention provides particles comprising chitosan, or a derivative thereof, useful as delivery vehicles for polynucleotides encoding polypeptides, compositions comprising such particles and a pharmaceutically acceptable carrier, and methods for delivering polynucleotides using such particles. Optionally, the particles of the invention further comprise a lipid component. The present invention further provides a method for enhancing interferon-gamma expression to regulate the production of cytokines secreted by T-helper type 2 (Th2) cells within a subject by administering an effective amount of a particle of the subject invention to the subject, wherein the particle comprises a polynucleotide encoding interferon-gamma.

### CROSS-REFERENCE TO RELATED APPLICATIONS

The present application claims benefit of U.S. Provisional Application Ser. No. 60/319,946, filed Feb. 14, 2003, and U.S. Provisional Application Ser. No. 60/319,956, filed Feb. 19, 2003, which are hereby incorporated by reference herein in their entirety, including any figures, tables, nucleic acid sequences, amino acid sequences, or drawings.

### BACKGROUND OF THE INVENTION

An elegant approach to in vivo gene expression involves the use of plasmid DNAs, pDNAs, which have a number of advantages, including ease of use and preparation, stability and heat resistance, and unlimited size. Plasmids do not replicate in mammalian hosts and do not integrate into host genomes; yet they can persist in host cells and express the cloned gene for a period of weeks to months. A major drawback of the pDNA approach is that gene transfer is inefficient under physiologically relevant conditions, especially in slow and non-dividing cells, such as epithelial cells. There is a need for the development of safer and more effective delivery vehicles, both for antigens and genes. The gene delivery systems should offer the freedom to manipulate the complex stoichiometry, surface charge density, and hydrophobicity needed for interaction with the cellular lipid components.

Cationic polymers and cationic phospholipids are the two major types of non-viral gene delivery vectors currently being investigated. Due to their permanent cationic charge, both types interact electrostatically with negatively charged DNA and form complexes (lipo- or polyplexes). Despite the ease of fabrication of the lipoplexes, their low transfection efficiency and toxicity limits their success. However, polyplexes involving cationic polymers are more stable than cationic lipids (De Smedt, S. C. et al. Pharm. Res., 2000, 17:113-126). Nevertheless, the transfection efficiency is relatively lower than that of viral vectors. The precise mechanism for gene transfection mediated by cationic liposomes is still unclear. However, fusion of endosomal and liposomal membranes or destabilization of the endosomal membrane by cationic liposomes may trigger cytosolic delivery of DNA (Koltover, T. et al. Science, 1998, 281:78-81).

Cationic polymers have been used to condense and deliver DNA both in vitro and in vivo. Several cationic polymers have been investigated that lead to higher transfection efficiencies (De Smedt, S. C. et al. Pharm. Res., 2000, 17:11-26; Garnett, M. C. Crit. Rev. Ther. Drug Carrier Syst., 1999, 16:147-207). They form polyelectrolyte complexes with plasmid DNA in which the DNA becomes better protected against nuclease degradation (Minagawa, K. et al. FEBS Lett., 1991, 295:67-69). They show structural variability and versatility including the possibility of covalent binding of the targeting moieties for gene expression mediated through specific receptors (De Smedt, S. C. et al. Pharm. Res., 2000, 17:131-126). Cationic liposomes form a complex with anionic DNA molecules and are thought to deliver DNA by endocytosis (Wrobell, D. et al. Biochem.Biophys.Acta, 1995, 1235:296-304). Polymeric gene carriers might have some advantages over liposome systems: (i) relatively small size and narrow distribution; (ii) high stability against nucleases; and (iii) easy control of the hydrophilicity of the complex by copolymerization (Kabanov, A. V. Pharm.Sci.Tech.Today, 1999, 2:265-372).

The best characterized chitin-based copolymer, chitosan, is a biodegradable and biocompatible natural biopolymer that increases nasal absorption of the drug without any adverse effects (Thanou, M. et al. Biomaterials 2002, 23:153-9; Kim, Y. H. et al. Bioconjug Chem, 2001, 12:932-8; Singla, A. K. et al. J Pharm Pharmacol, 2001, 53:1047-67; Brooking, J, et al. J Drug Target, 2001, 9:267-79; Kotze, A. F. et al. J Pharm Sci, 1999, 88:253-7; van der Lubben, I. M. et al. Eur J Pharm Sci, 2001, 14:201-7). A major stumbling block in in vivo gene expression systems has been the lack of efficient transfection in vivo, and the improvements have been empirical.

Chitosan, a natural, biocompatible cationic polysaccharide prepared from crustacean shells, has shown much potential as a vehicle for gene delivery. Chitosan has many beneficial effects, including immunostimulatory activity (Nishimura, K. et al. Vaccine, 1984, 2:93-9), anticoagulant activity (Otterlei, M. et al. Vaccine, 1994, 12:825-32), wound-healing properties (Muzzarelli, R. et al. Biomaterials, 1988, 10:589-603), and anti-microbial properties (Pappineau, A. M. et al. Food Biotechnol, 1991, 5:45-47). Additionally, chitosan is non-toxic, non-hemolytic, weakly immunogenic, slowly biodegradable, and nuclease resistant; and it has been used in controlled drug delivery (Erbacher, P. et al. Pharm Res, 1998, 15:1332-9; Richardson, S. C. et al. Int J Pharm, 1999, 178:231-43). Chitosan increases transcellular and paracellular transport across the mucosal epithelium and thus may facilitate mucosal gene delivery and the immune responsiveness of the mucosa and bronchus-associated lymphoid tissue. Therefore, chitosan appears to possess the attributes for an ideal gene delivery agent required for therapies such as lung disease therapy.

IFN-γ, a pleiotropic cytokine, promotes T-helper type-1 (Th1) responses, which downregulate the Th2-like immune responses that are hallmarks of allergic diseases, including asthma (Mosman, T. R. et al. Ann Rev Immunol, 1989, 7:145-173; Umetsu, D. T. et al. J Allergy Clin Immunol, 1997, 100:1-6). Administration of recombinant IFN-γ reverses established airway disease and inflammation in murine models (Flaishon, L. et al. J Immunol, 2002, 168:3707-11; Yoshida, M. et al. Am J Respir Crit Care Med, 2002, 166:451-6). Application of IFN-γ for treatment of asthma has been limited because of the short half-life of IFN-γ in vivo and the potentially severe adverse effects associated with high dose administration (Murray, H. Intensive Care Med, 1997, 22(Suppl 4):S456-61). IFN-γ gene transfer inhibits both antigen- and Th2-induced pulmonary eosinophilia and airway hyperreactivity (Li, X. M. et al. J Immunol, 1996, 157:3216-9; Dow, S. W. et al. Hum Gene Ther, 1999, 10:1905-14). However, those results are not directly applicable to humans because of the methods used in the investigations, such as the intratracheal administration or injection of DNA with lipofectamine. Moreover, the direct effects of these cytokine plasmids as therapeutics for allergic asthma have not been addressed. A major drawback of the pDNA approach is that gene transfer is inefficient under physiologically permissible conditions, especially in non-dividing cells such as epithelial cells.

The protective role of IFN-γ gene transfer in a mouse model for respiratory syncytial virus infection (U.S. Pat. No. 6,489,306 (Mohapatra et al., issued Dec. 3, 2002); Kumar, M. Vaccine, 1999, 18:558-567) and the role of IFN-γ as a genetic adjuvant in the immunotherapy of grass-allergic asthma (Kumar, M. et al. J Allergy Clin Immunol, 2001, 108:402-408) has previously been reported. IFN-γ is considered to be a prime candidate for asthma therapy because of its capacity to decrease: (i) IL-13-induced goblet cell hyperplasia and eosinophilia by upregulation of the IL-13Rα2 decoy receptor, which diminishes IL-13 signaling (Ford, J. G. et al. J Immunol, 2001, 167:1769-1777; Daines, M. O. and Hershey, G. K. J Biol Chem 2002, 277(12):10387-10393); (ii) LTC4 production in murine and human macrophages (Boraschi, D. et al. J Immunol, 1987, 138:4341-4346; Thivierge, M. et al. J Immunol, 2001, 167:2855-2860), in human peripheral blood lymphocytes after wasp venom immunotherapy (Pierkes, M. et al. J Allergy Clin Immunol, 1999, 103:326-332), and in leukocytes of pollinosis patients (Krasnowska, M. et al. Arch Immunol Ther Exp (Warsz), 2000, 48:287-292); and (iii) TGF-β and procollagen-I and -III, which cause fibrosis and airway remodeling (Gurujeyalakshmi, G. et al. Exp Lung Res, 1995, 21:791-808; Minshall, E. et al. Am J Respir Cell Mol Biol, 1997, 17:326-333).

This disclosure demonstrates that the gene transfer efficiency can be significantly increased using a novel improved formulation of hybrid nanoparticles, referred to as Chlipids. Further, therapy with chitosan-IFN-gamma gene-nanoparticles carrying (CIN) constitutes a novel non-viral approach to mucosal gene transfer for asthma. CIN therapy significantly inhibits the production of IL-4, IL-5, ovalbumin (OVA)-specific serum IgE, airway inflammation, and hyperreactivity in a BALB/c mouse model of allergic asthma.

### BRIEF SUMMARY OF THE INVENTION

The present invention pertains to gene delivery systems using chitosan, or derivatives thereof. In one aspect, the present invention provides particles comprising chitosan, or a derivative thereof, useful as delivery vehicles for polynucleotides, compositions comprising such particles and a pharmaceutically acceptable carrier, and methods for delivering and expressing polynucleotides to hosts in vitro or in vivo using such particles. Optionally, the particles of the invention further comprise a lipid component and are referred to herein interchangeably as “chliposomes” or “chlipids” or “chitosan-lipid nanoparticles” or “CLNs”. The invention further includes methods for producing particles of the subject invention.

The present further provides a method for enhancing interferon-gamma expression to regulate the production of cytokines secreted by T-helper type 2 (Th2) cells within a subject by administering an effective amount of a particle of the subject invention to the subject, wherein the particle comprises a polynucleotide encoding interferon-gamma.

### BRIEF DESCRIPTION OF THE DRAWINGS

For a fuller understanding of the nature and objects of the invention, reference should be made to the following detailed description, taken in connection with the accompanying drawings, in which:

FIGS. 1A-1C show optimization protocols of combining chitosan and lipids for gene transfer. FIG. 1A shows the DNA recovery from pelleted chlipids. FIG. 1B shows the optimal lipid concentration. FIG. 1C shows the optimal serum concentration.

FIGS. 2A-2C show electron micrographs of nanoparticles. FIG. 2A shows chitosan at 14,000× magnification. FIG. 2B shows lipid-DNA at 7,000× magnification. FIG. 2C shows chitosan+(lipid-DNA) at 44,000× magnification.

FIGS. 3A-3C show distribution and quantification of transfection of the GFP gene lung cells. The green fluorescence seen in the lung section suggests that the epithelial cells are predominantly transfected by chitosan-lipid nanoparticle (CLN) (FIG. 3A). The cells from the BAL fluid showed that monocytes are also transfected and express GFP (FIG. 3B). In FIG. 3C, “1” is chitosan, “2” is lipofectin, “3” is CLNs, and “4” is DNA alone. The quantification of EGFP-positive BAL cells showed that while chitosan and LIPOFECTIN showed a similar transfection efficiency (20%) in vivo, CLN showed significantly higher (30%, P<0.05) transfection efficiency, as shown in FIG. 3C.

FIG. 4 shows quantification of IL-6 in bronchioalveolar fluid (BAL) following intranasal administration of nanoparticle. Quantification of IL-6 showed that CLN-DNA nanoparticles induced significantly decreased IL-6 levels compared to chitosan-pVAX complexes.

FIGS. 5C-5C show that chitosan particles target lung epithelial cells and monocytes. BALB/c mice were administered with chitosan particles containing pVAX-GFP. After 24 hours, mice were sacrificed and their lungs were fixed and sectioned by cryotome. Sections (15 microns) were thaw-mounted to slides and sections were viewed for green fluorescent protein under a microscope and photographed (“Lung”; FIG. 5A). BAL cells were fixed after cytospin on a slide and visualized under a fluorescent microscope to identify GFP expressing cells (“BAL”; FIG. 5B). FIG. 5C is a graph showing that chitosan IFN-gamma-pDNA nanoparticle (CIN) administration induces IFN-γ production in the lung over a period of 10 days. Lung homogenates were prepared from mice after 1, 2, 4, 6, 8, or 10 days of treatment with CIN (25 μg/mouse) or chitosan alone, and IFN-γ levels were determined by ELISA (n=3).

FIGS. 6A-6F show prevention of airway hyperresponsiveness (AHR). FIG. 6A shows a schematic prophylaxis protocol. Mice were challenged with methacholine on day 22 to measure airway responsiveness (FIG. 6B). The values are mean enhanced pause (PENH) expressed as percent of baseline±SEM (*P<0.05, **P<0.01). On day 24, BAL was performed and differential cell count was obtained (FIG. 6C). On day 24, lungs were removed, sectioned, and the sections stained with hematoxylin/eosin (“PBS, phosphate-buffered saline control; “N-DNA”, naked DNA without chitosan; “CIN”, chitosan-DNA complex), as shown in FIGS. 6D, 6E, and 6F. Differential cell counts and examination of tissue sections were performed by different persons in a blinded fashion. Representative results are shown.

FIGS. 7A-7C show that CIN alters production of cytokines and IgE. On day 23 of the prophylactic procedure (see schematic of FIG. 6A), spleens ere removed and single-cell suspensions of splenocytes were prepared. Cells were cultured for 48 hours with ovalbumin (OVA) and the levels of secreted IFN-γ and IL-5 (FIG. 7A) and IL-4 (FIG. 7B) were measured. Total serum IgE was measured on day 23 (FIG. 7C). Values are means±SEM (*p<0.05, **p<0.01).

FIGS. 8A-8D show reversal of established AHR and eosinophilia. FIG. 8A shows a schematic of the therapeutic protocol. Mice were sensitized (i.p.) and challenged (i.n.) with OVA and treated with CIN as described. AHR was measured 24 hours after the last challenge (n=4). CIN-treated mice exhibited reduced AHR compared to the controls (FIG. 8B). Data are mean enhanced pause (PENH) expressed as percent of baseline±SEM (*p<0.05). On day 31, BAL was performed and eosinophils in BAL fluid were counted (**p<0.01). FIG. 8C shows that CIN therapy decreases eosinophils. On day 23, spleens were removed and single-cell suspensions of splenocytes were prepared. Cells were cultured for 48 hours in the presence of OVA and cell supernatants were analyzed for IFN-γ, IL-4, and IL-5. Mice receiving CIN showed more production of IFN-γ and less IL-4 and IL-5 compared to the chitosan-only control (FIG. 8D). Data are means±SEM (*p<0.05).

FIGS. 9A-9D show that CIN treatment induces apoptosis of goblet cells. BALB/c mice (n=3) were sensitized and challenged with OVA as in FIGS. 8A, and then treated with intranasal CIN therapy. Mice were sacrificed at 0, 3, 6, or 12 hours after CIN treatment and lungs were removed, sectioned and stained with hematoxylin/eosin (FIGS. 9A-9D, respectively).

FIGS. 10A-10D show that CIN treatment induced apoptosis of goblet cells. BALB/c mice (n=3) were sensitized and challenged with OVA as in FIGS. 8A, and then treated with intranasal CIN therapy. Mice were sacrificed at 0, 3, 6, or 12 hours after CIN treatment and lungs were removed, sectioned, and analyzed for apoptosis by TUNEL (terminal dUTP nick end labeling) assay (FIGS. 10A-10D, respectively).

FIGS. 11A-11C show a final set of lung sections from FIG. 10B (6-hour time point) stained for the goblet cell-specific Muc5a (FIG. 11C), and for apoptosis by the TUNEL assay (FIG. 11B). FIG. 11A shows staining of nuclei with diamidinophenylindole (DAPI).

FIGS. 12A-12C show that CIN therapy involves the STAT4 pathway. OVA-sensitized BALB/c wild-type (WT) and STAT 4^{-/-} knockout mice (n=4) were given CIN therapy intranasally and challenged with OVA. AHR in response to methacholine was measured one day after the last challenge (FIG. 12A). The values are means±SEM (*p<0.05). Mice were sacrificed the day following AHR measurement and their lungs were removed, paraffin-embedded and stained with hematoxylin/eosin (FIGS. 12B and 12C).

### DETAILED DISCLOSURE OF THE INVENTION

The present invention provides particles comprising chitosan, or a derivative thereof; and a polynucleotide. Preferably, the particle further comprises a control sequence operably-linked to the polynucleotide, which is capable of causing expression of the polynucleotide within a host in vitro or in vivo. The present invention further provides compositions comprising a particle of the present invention and a pharmaceutically acceptable carrier.

Optionally, the particle of the present invention comprises a lipid that is complexed with the chitosan and the polynucleotide component of the particle. Since efficient gene expression in vivo requires both complex formation for cell uptake and prevention of nucleotide degradation and complex dissociation for transcription by RNA polymerase, the present inventor hypothesized that a combination of both chitosan and liposomes may lead to increased gene delivery and expression in vivo. Therefore, the present inventor has developed methods that combine these two different carrier systems to develop a novel gene delivery system designated “chliposomes” that exhibits a significant increase in gene DNA transfection and gene expression (also referred to herein as “chlipids” and used interchangeably). Preferably, the components of the chlipid are oriented such that the polynucleotide is surrounded by a lipid monolayer, with polynucleotide-lipid inverted cylindrical micelles arranged in a hexagonal lattice.

The present invention further includes a method for producing the particles of the invention by mixing (e.g., complexing) a polynucleotide and chitosan or a chitosan derivative, to form a particle comprising a binary complex of the polynucleotide and the chitosan or chitosan derivative. Optionally, the method further comprises mixing (complexing) a lipid with the polynucleotide and chitosan or chitosan derivative to form a particle (chlipid) comprising a multiplex of the polynucleotide, chitosan or chitosan derivative, and the lipid. Typically, the particles of the present invention range in size from the nanometer range (e.g., less than one micrometer; nanoparticles) to the micrometer size range (e.g., about one micrometer or larger).

The type of reaction vessel or vessels utilized for producing the particles of the present invention, or their sizes, are not critical. Any vessel or substrate capable of holding or supporting the reactants so as to allow the reaction to take place can be used. It should be understood that, unless expressly indicated to the contrary, the terms “adding”, “contacting”, “mixing”, “reacting”, “combining” and grammatical variations thereof, are used interchangeably to refer to the mixture of reactants of the method of the present invention (e.g., polynucleotide or non-polynucleotide agent, chitosan or chitosan derivative, lipid, and so forth), and the reciprocal mixture of those reactants, one with the other (i.e., vice-versa), in any order.

It will be readily apparent to those of ordinary skill in the art that a number of general parameters can influence the efficiency of transfection or polynucleotide delivery. These include, for example, the concentration of polynucleotide to be delivered, the concentration of chitosan or chitosan derivative, and the concentration of lipid (for chlipids of the present invention). For in vitro delivery, the number of cells transfected, the medium employed for delivery, the length of time the cells are incubated with the particles of the invention, and the relative amount of particles can influence delivery efficiency. For example, a 1:5 ratio of polynucleotide to lipid, 1:5 ratio of polynucleotide to chitosan, and 20% serum is suitable. These parameters can be optimized for particular cell types and conditions. Such optimization can be routinely conducted by one of ordinary skill in the art employing the guidance provided herein and knowledge generally available to those skilled in the art. It will also be apparent to those of ordinary skill in the art that alternative methods, reagents, procedures and techniques other than those specifically detailed herein can be employed or readily adapted to produce the particles and compositions of the invention. Such alternative methods, reagents, procedures and techniques are within the spirit and scope of this invention.

In another aspect, the present invention provides a method for delivery and expression of a polynucleotide within a host or subject by administering a particle of the present invention to the host or subject. Optionally, the polynucleotide encodes a polypeptide. The polypeptide encoded by the polynucleotide of the particle can be a hormone, receptor, enzyme, or other desired polypeptide. For example, the polypeptide can comprise a cytokine, such as interferon-gamma. The polypeptide may serve a therapeutic and/or diagnostic purpose, for example. In other embodiments, the polynucleotide does not encode a polypeptide. The polynucleotide may comprise interfering RNA, for example.

In another aspect, the present invention provides a method for enhancing interferon-gamma expression to regulate the production of cytokines secreted by T-helper type 2 (Th2) cells within a subject by administering an effective amount of a particle to the subject, wherein the particle comprises chitosan, or a derivative thereof, and a polynucleotide encoding interferon-gamma Preferably, the particle is administered to the respiratory tract of the subject. In one embodiment, the subject is suffering from asthma. In another embodiment, the subject is not suffering from asthma. Preferably, the particle administered to the subject is a chlipid of the present invention.

The method of the subject invention for enhancing interferon-gamma expression to regulate the production of cytokines secreted by Th2 cells (such as IL-4 and/or IL-5) within a subject preferably results in inhibition of airway inflammation and airway hyperresponsiveness (AHR), the hallmarks of allergic asthma, when administered to the subject.

The term “chitosan”, as used herein, will be understood by those skilled in the art to include all derivatives of chitin, or poly-N-aceryl-D-glucosamine (including all polyglucosamine and oligomers of glucosamine materials of different molecular weights), in which the greater proportion of the N-acetyl groups have been removed through hydrolysis. Generally, chitosans are a family of cationic, binary hetero-polysaccharides composed of (1→4)-linked 2-acetamido-2-deoxy-β-D-glucose (GlcNAc, A-unit) and 2-amino-2-deoxy-β-D-glucose, (GlcN; D-unit) (Varum K. M. et al., Carbohydr. Res., 1991, 217:19-27; Sannan T. et al., Macromol. Chem., 1776, 177:3589-3600). Preferably, the chitosan has a positive charge. Chitosan, chitosan derivatives or salts (e.g. nitrate, phosphate, sulphate, hydrochloride, glutamate, lactate or acetate salts) of chitosan may be used and are included within the meaning of the term “chitosin”. As used herein, the term “chitosan derivatives” are intended to include ester, ether or other derivatives formed by bonding of acyl and/or alkyl groups with OH groups, but not the NH₂ groups, of chitosan. Examples are O-alkyl ethers of chitosan and O-acyl esters of chitosan. Modified chitosans, particularly those conjugated to polyethylene glycol, are included in this definition. Low and medium viscosity chitosans (for example CL113, G210 and CL110) may be obtained from various sources, including PRONOVA Biopolymer, Ltd. (UK); SEIGAGAKU America Inc. (Maryland, USA); MERON (India) Pvt, Ltd. (India); VANSON Ltd. (Virginia, USA); and AMS Biotechnology Ltd. (UK). Suitable derivatives include those which are disclosed in Roberts, Chitin Chemistry, MacMillan Press Ltd., London (1992). Optimization of structural variables such as the charge density and molecular weight of the chitosan for efficiency of polynucleotide delivery and expression is contemplated and encompassed by the present invention.

The chitosan (or chitosan derivative or salt) used preferably has a molecular weight of 4,000 Dalton or more, preferably in the range 25,000 to 2,000,000 Dalton, and most preferably about 50,000 to 300,000 Dalton. Chitosans of different low molecular weights can be prepared by enzymatic degradation of chitosan using chitosanase or by the addition of nitrous acid. Both procedures are well known to those skilled in the art and are described in various publications (Li et al., Plant Physiol. Biochem., 1995, 33: 599-603; Allan and Peyron, Carbohydrate Research, 1995, 277:257-272; Damard and Cartier, Int. J. Biol. Macromol., 1989, 11: 297-302). Preferably, the chitosan is water-soluble and may be produced from chitin by deacetylation to a degree of greater than 40%, preferably between 50% and 98%, and more preferably between 70% and 90%.

The lipid utilized for the particles, compositions, and methods of the present invention is preferably a phospholipid or cationic lipid. Cationic lipids are amphipathic molecules, containing hydrophobic moieties such as cholesterol or alkyl side chains and a cationic group, such as an amine. Phospholipids are amphipathic molecules containing a phosphate group and fatty acid side chains. Phospholipids can have an overall negative charge, positive charge, or neutral charge, depending on various substituents present on the side chains. Typical phospholipid hydrophilic groups include phosphatidyl choline, phosphatidylglycerol, and phosphatidyl ethanolamine moieties. Typical hydrophobic groups include a variety of saturated and unsaturated fatty acid moieties. The lipids used in the present invention include cationic lipids that form a complex with the genetic material (e.g., polynucleotide), which is generally polyanionic, and the chitosan or chitosan derivative. The lipid may also bind to polyanionic proteoglycans present on the surface of cells. The cationic lipids can be phospholipids or lipids without phosphate groups.

A variety of suitable cationic lipids are known in the art, such as those disclosed in International Publication No. WO 95/02698, the disclosure of which is herein incorporated by reference in its entirety. Exemplified structures of cationic lipids useful in the particles of the present invention are provided in Table 1 of International Publication No. WO 95/02698. Generally, any cationic lipid, either monovalent or polyvalent, can be used in the particles, compositions and methods of the present invention. Polyvalent cationic lipids are generally preferred. Cationic lipids include saturated and unsaturated allyl and alicyclic ethers and esters of amines, amides or derivatives thereof. Straight-chain and branched alkyl and alkene groups of cationic lipids can contain from 1 to about 25 carbon atoms. Preferred straight-chain or branched alkyl or alkene groups have six or more carbon atoms. Alicyclic groups can contain from about 6 to 30 carbon atoms. Preferred alicyclic groups include cholesterol and other steroid groups. Cationic lipids can be prepared with a variety of counterions (anions) including among others: chloride, bromide, iodide, fluoride, acetate, trifluoroacetate, sulfate, nitrite, and nitrate.

Transfection efficiency can be increased by using a lysophosphatide in particle formation. Preferred lysophosphatides include lysophosphatidylcholines such as I-oleoyllysophosphatidylcholine and lysophosphatidylethanolamines. Well known lysophosphatides which may be used include DOTMA (dioleyloxypropyl trimethylammonium chloride/DOPE (i.e., LIPOFECTIN, GIBCO/BRL, Gaithersburg, Md.), DOSPA, (dioleyloxy sperminecarboxamidoethyl dimethylpropanaminium trifuoroacetate)/DOPE (i.e., LIPOFECTAMINE), LIPOFECTAMINE 2000, and DOGS (dioctadecylamidospermine) (i.e., TRANSFECTAM), and are all commercially available. Additional suitable cationic lipids structurally related to DOTMA are described in U.S. Pat. No. 4,897,355, which is herein incorporated by reference in its entirety.

TRANSFECTAM belongs to a group of cationic lipids called lipopolamines (also referred to as second-generation cationic lipids) that differ from the other lipids used in gene transfer mostly by their spermine head group. The polycationic spermine head group promotes the formation of lipoplexes with better-defined structures (e.g., 50 to 100 nm) (Remy J. S. et al., “Gene Transfer with Lipospermines and Polyethylenimines”, Adv. Drug Deliv. Rev., 1998, 30:85-95).

Another useful group of cationic lipids related to DOTMA and DOTAP are commonly called DORI-ethers or DORI-esters, such as (DL-1-O-oleyl-2-oleyl-3-dimethylaminopropyl-β-hydroxyethylammonium or DL-1-oleyl-2-O oleyl-3-dimethyl-aminopropyl-β-hydroxyethylammonium). DORI lipids differ from DOTMA and DOTAP in that one of the methyl groups of the trimethylammonium group is replaced with a hydroxyethyl group. The oleoyl groups of DORI lipids can be replaced with other alkyl or alkene groups, such as palmitoyl or stearoyl groups. The hydroxyl group of the DORI-type lipids can be used as a site for further functionalization, for example for esterification to amines, like carboxyspermine. Additional cationic lipids which can be employed in the particles, compositions, and methods of the present invention include those described in International Publication No. WO 91/15501, which is herein incorporated by reference in its entirety. Cationic sterol derivatives, like 3 β[N-(N′,N′-dimethylaminoeth-ane)carbamoyl] cholesterol (DC-Chol) in which cholesterol is linked to a trialkyammonium group, can also be employed in the present invention. DC-Chol is reported to provide more efficient transfection and lower toxicity than DOTMA-containing liposomes for some cell lines. DC-Chol polyamine variants such as those described in International Publication No. WO 97/45442 may also be used. Polycationic lipids containing carboxyspermine are also useful in the delivery vectors or complexes of this invention. EP-A-304111 describes carboxyspermine containing cationic lipids including 5-carboxyspermylglycine dioctadecyl-amide (DOGS), as referenced above, and dipalmitoylphosphatidylethanolamine 5-carboxyspermylamide (DPPES). Additional cationic lipids can be obtained by replacing the octadecyl and palmitoyl groups of DOGS and DPPES, respectively, with other alkyl or alkene groups. Cationic lipids can optionally be combined with non-cationic co-lipids, preferably neutral lipids, to form the chlipids of the invention. One or more amphiphilic compounds can optionally be incorporated in order to modify the particle's surface property.

Suitable cationic lipids include esters of the Rosenthal Inhibitor (RI) (DL-2,3-distearoyloxypropyl(dimethyl)-β-hydroxyethylammoniumbromide), as described in U.S. Pat. No. 5,264,618, the contents of which is hereby incorporated by reference in its entirety. These derivatives can be prepared, for example, by acyl and alkyl substitution of 3-dimethylaminopropane diol, followed by quaternization of the amino group. Analogous phospholipids can be similarly prepared.

The particles of the present invention can be targeted through various means. The size of the particle provides one means for targeting to cells or tissues. For example, relatively small particles efficiently target ischemic tissue and tumor tissue, as described in U.S. Pat. No. 5,527,538, and U.S. Pat. Nos. 5,019,369, 5,435,989 and 5,441,745, the contents of which are hereby incorporated by reference in their entirety.

The particles of the invention can be targeted according to the mode of administration. For example, lung tissue can be targeted by intranasal administration, cervical cells can be targeted by intravaginal administration, and prostate tumors can be targeted by intrarectal administration. Skin cancer can be targeted by topical administration. Depending on location, tumors can be targeted by injection into the tumor mass.

Further, particles of the invention can be targeted by incorporating a ligand such as an antibody, a receptor, or other compound known to target particles such as liposomes or other vesicles to various sites. The ligands can be attached to cationic lipids used to form the particles of the present invention, or to a neutral lipid such as cholesterol used to stabilize the particle. Ligands that are specific for one or more specific cellular receptor sites are attached to a particle to form a delivery vehicle that can be targeted with a high degree of specificity to a target cell population of interest.

Suitable ligands for use in the present invention include, but are not limited to, sugars, proteins such as antibodies, hormones, lectins, major histocompatibility complex (MHC), and oligonucleotides that bind to or interact with a specific site. An important criteria for selecting an appropriate ligand is that the ligand is specific and is suitably bound to the surface of the particles in a manner which preserves the specificity. For example, the ligand can be covalently linked to the lipids used to prepare the particles. Alternatively, the ligand can be covalently bound to cholesterol or another neutral lipid, where the ligand-modified cholesterol is used to stabilize the lipid monolayer or bilayer.

IFN-γ is a 14-18 kDalton 143 amino acid glycosylated protein that is a potent multifunctional cytokine. As used herein, “interferon-gamma”, “IFN-gamma”, “interferon-γ”, and “IFN-γ refer to IFN-γ protein, biologically active fragments of IFN-γ, and biologically active homologs of “interferon-gamma” and “IFN-γ”, such as mammalian homologs. These terms include IFN-γ-like molecules. An “IFN-γ-like molecule” refers to polypeptides exhibiting IFN-γ-like activity when the polynucleotide encoding the polypeptide is expressed, as can be determined in vitro or in vivo. For purposes of the subject invention, IFN-γ-like activity refer to those polypeptides having one or more of the functions of the native IFN-γ cytokine, such as those disclosed herein. Fragments and homologs of IFN-γ retaining one or more of the functions of the native IFN-γ cytokine, such as those disclosed herein, is included within the meaning of the term “IFN-γ”. In addition, the term includes a nucleotide sequence which through the degeneracy of the genetic code encodes a similar peptide gene product as IFN-γ and has the IFN-γ activity described herein. For example, a homolog of “interferon-gamma” and “IFN-γ” includes a nucleotide sequence which contains a “silent” codon substitution (e.g., substitution of one codon encoding an amino acid for another codon encoding the same amino acid) or an amino acid sequence which contains a “silent” amino acid substitution (e.g., substitution of one acidic amino acid for another acidic amino acid). An exemplified nucleotide sequence encodes human IFN-γ (Accession No: NM_{—}000639, NCBI database, which is hereby incorporated by reference in its entirety).

The polynucleotides are administered and dosed in accordance with good medical practice, taking into account the clinical condition of the individual patient, the site and method of administration, scheduling of administration, patient age, sex, body weight, and other factors known to medical practitioners. The therapeutically or pharmaceutically “effective amount” for purposes herein is thus determined by such considerations as are known in the art. A therapeutically or pharmaceutically effective amount of nucleic acid molecule (such as an IFN-γ-encoding polynucleotide) is that amount necessary to provide an effective amount of the polynucleotide, or the corresponding polypeptide(s) when expressed in vivo. An effective amount of an agent, such as a polynucleotide or non-polynucleotide agent, or particles comprising such polynucleotide or non-polynucleotide agents, can be an amount sufficient to prevent, treat, reduce and/or ameliorate the symptoms and/or underlying causes of any pathologic condition, such as a disease or other disorder. In some instances, an “effective amount” is sufficient to eliminate the symptoms of the pathologic condition and, perhaps, overcome the condition itself. In the context of the present invention, the terms “treat” and “therapy” and the like refer to alleviate, slow the progression, prophylaxis, attenuation, or cure of existing condition. The term “prevent”, as used herein, refers to putting off, delaying, slowing, inhibiting, or otherwise stopping, reducing, or ameliorating the onset of such conditions.

In the method of the invention for enhancing interferon-gamma expression, the amount of the polypeptide (IFN-γ) is preferably effective to achieve regulation of one or more cytokines secreted by Th2 cells, such as interleukin-4 (IL-4). The amount of IFN-γ may be sufficient to achieve inhibition of (Th2)-associated airway inflammation and airway hyperresponsiveness when administered to a subject. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

Mammalian species which benefit from the disclosed particles, compositions, and methods include, and are not limited to, apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales.

As used herein, the term “patient”, “subject”, and “host” are used herein interchangeably and intended to include such human and non-human mammalian species and cells of those species. For example, the term “host” includes one or more host cells, which may be prokaryotic (such as bacterial cells) or eukaryotic cells (such as human or non-human mammalian cells), and may be in an in vivo or in vitro state. In those cases wherein the polynucleotide utilized is a naturally occurring nucleic acid sequence, the polynucleotide encoding the polypeptide product can be administered to subjects of the same species or different species from which the nucleic acid sequence naturally exists, for example.

The particles of the present invention (and compositions containing them) can be administered to a subject by any route that results in delivery and/or expression of the genetic material (e.g., polynucleotides) or delivery of other non-polynucleotide agents carried by the particles. For example, the particles can be administered intravenously (I.V.), intramuscularly (I.M.), subcutaneously (S.C.), intradermally (I.D.), orally, intranasally, etc.

Examples of intranasal administration can be by means of a spray, drops, powder or gel and also described in U.S. Pat. No. 6,489,306, which is incorporated herein by reference in its entirety. One embodiment of the present invention is the administration of the invention as a nasal spray. Alternate embodiments include administration through any oral or mucosal routes such as oral, sublingual, intravaginal or intraanal administration, and even eye drops. However, other means of drug administrations such as subcutaneous, intravenous, and transdermal are well within the scope of the present invention.

The term “polynucleotide”, as used herein, refers to a polymeric form of nucleotides of any length, either ribonucleotides or deoxyribonucleotides. This term refers only to the primary structure of the molecule. Thus, the term includes double-stranded and single-stranded DNA, as well as double-stranded and single-stranded RNA. Thus, the term includes DNA, RNA, or DNA-DNA, DNA-RNA, or RNA-RNA hybrids, or protein nucleic acids (PNAs) formed by conjugating bases to an amino acid backgone. It also includes modifications, such as by methylation and/or by capping, and unmodified forms of the polynucleotide. The nucleotides may be synthetic, or naturally derived, and may contain genes, portions of genes, or other useful polynucleotides. In one embodiment, the polynucleotide comprises DNA containing all or part of the coding sequence for a polypeptide, or a complementary sequence thereof, such as interferon gamma. An encoded polypeptide may be intracellular, i.e., retained in the cytoplasm, nucleus, or in an organelle, or may be secreted by the cell. For secretion, the natural signal sequence present in a polypeptide may be retained. When the polypeptide or peptide is a fragment of a protein, a signal sequence may be provided so that, upon secretion and processing at the processing site, the desired protein will have the natural sequence. Specific examples of coding sequences of interest for use in accordance with the present invention include the polypeptide-coding sequences disclosed herein. The polynucleotides may also contain, optionally, one or more expressible marker genes for expression as an indication of successful transfection and expression of the nucleic acid sequences contained therein.

The polynucleotides may also be oligonucleotides, such as antisense oligonucleotides, chimeric DNA-RNA polymers, ribozymes, as well as modified versions of these nucleic acids wherein the modification may be in the base, the sugar moiety, the phosphate linkage, or any combination thereof.

Antisense oligonucleotides of the particles of the invention may be constructed to inhibit expression of a target gene. An antisense sequence can be wholly or partially complementary to a target nucleic acid, and can be DNA, or its RNA counterpart. Antisense nucleic acids can be produced by standard techniques (see, for example, Shewmaker et al., U.S. Pat. No. 5,107,065, issued Apr. 21, 1992). Antisense oligonucleotides may comprise a sequence complementary to a portion of a protein coding sequence. A portion, for example a sequence of 16 nucleotides, may be sufficient to inhibit expression of the protein. An antisense nucleic acid sequence or oligonucleotide complementary to 5′ or 3′ untranslated regions, or overlapping the translation initiation codons (5′ untranslated and translated regions), of target genes, or genes encoding a functional equivalent can also be effective. Accordingly, antisense nucleic acids or oligonucleotides can be used to inhibit the expression of the gene encoded by the sense strand or the mRNA transcribed from the sense strand. In addition, antisense nucleic acids and oligonucleotides can be constructed to bind to duplex nucleic acids either in the genes or the DNA:RNA complexes of transcription, to form stable triple helix-containing or triplex nucleic acids to inhibit transcription and/or expression of a gene (Frank-Kamenetskii, M. D. and Mirkin, S. M., 1995, Ann. Rev. Biochem. 64:65-95). Such oligonucleotides can be constructed using the base-pairing rules of triple helix formation and the nucleotide sequences of the target genes.

According to the present invention, an isolated nucleic acid molecule or nucleic acid sequence is a nucleic acid molecule or sequence that has been removed from its natural milieu. As such, “isolated” does not necessarily reflect the extent to which the nucleic acid molecule has been purified.

The terms “polypeptide” and “protein” are used interchangeably herein and indicate a molecular chain of amino acids of any length linked through peptide bonds. Thus, peptides, oligopeptides, and proteins are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

The particles of the present invention are useful as vectors for the delivery of polynucleotides to hosts in vitro or in vivo. The term “vector” is used to refer to any molecule (e.g., nucleic acid or plasmid) usable to transfer a polynucleotide, such as coding sequence information (e.g., nucleic acid sequence encoding a protein or other polypeptide), to a host cell. A vector typically includes a replicon in which another polynucleotide segment is attached, such as to bring about the replication and/or expression of the attached segment. The term includes expression vectors, cloning vectors, and the like. Thus, the term includes gene expression vectors capable of delivery/transfer of exogenous nucleic acid sequences into a host cell. The term “expression vector” refers to a vector that is suitable for use in a host cell (e.g. a subject's cell, tissue culture cell, cells of a cell line, etc.) and contains nucleic acid sequences which direct and/or control the expression of exogenous nucleic acid sequences. Expression includes, but is not limited to, processes such as transcription, translation, and RNA splicing, if introns are present. Nucleic acid sequences can be modified according to methods known in the art to provide optimal codon usage for expression in a particular expression system. The vector of the present invention may include elements to control targeting, expression and transcription of the nucleic acid sequence in a cell selective manner as is known in the art. The vector can include a control sequence, such as a promoter for controlling transcription of the exogenous material and can be either a constitutive or inducible promoter to allow selective transcription. The expression vector can also include a selection gene.

A “coding sequence” is a polynucleotide sequence that is transcribed into mRNA and/or translated into a polypeptide. The boundaries of the coding sequence are determined by a translation start codon at the 5′-terminus and a translation stop codon at the 3′-terminus. A coding sequence can include, but is not limited to, mRNA, cDNA, and recombinant polynucleotide sequences. Variants or analogs may be prepared by the deletion of a portion of the coding sequence, by insertion of a sequence, and/or by substitution of one or more nucleotides within the sequence. For example, the particles of the present invention may be used to deliver coding sequences for interferon gamma, or variants or analogs thereof. Techniques for modifying nucleotide sequences, such as site-directed mutagenesis, are well known to those skilled in the art (See, e.g. Sambrook et al., Molecular Cloning: A Laboratory Manual, Second Edition, 1989; DNA Cloning, Vols. I and II, D. N. Glover ed., 1985). Optionally, the polynucleotides used in the particles of the present invention, and composition and methods of the invention that utilize such particles, can include non-coding sequences.

The term “operably-linked” is used herein to refer to an arrangement of flanking control sequences wherein the flanking sequences so described are configured or assembled so as to perform their usual function. Thus, a flanking control sequence operably-linked to a coding sequence may be capable of effecting the replication, transcription and/or translation of the coding sequence under conditions compatible with the control sequences. For example, a coding sequence is operably-linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence, and the promoter sequence can still be considered “operably-linked” to the coding sequence. Each nucleotide sequence coding for a polypeptide will typically have its own operably-linked promoter sequence. The promoter can be a constitutive promoter, or an inducible promoter to allow selective transcription. Optionally, the promoter can be a cell-specific or tissue-specific promoter. Promoters can be chosen based on the cell-type or tissue-type that is targeted for delivery or treatment, for example.

The terms “transfection” and “transformation” are used interchangeably herein to refer to the insertion of an exogenous polynucleotide into a host, irrespective of the method used for the insertion, the molecular form of the polynucleotide that is inserted, or the nature of the host (e.g., prokaryotic or eukaryotic). The insertion of a polynucleotide per se and the insertion of a plasmid or vector comprised of the exogenous polynucleotide are included. The exogenous polynucleotide may be directly transcribed and translated by the host or host cell, maintained as a nonintegrated vector, for example, a plasmid, or alternatively, may be stably integrated into the host genome. The terms “administration” and “treatment” are used herein interchangeably to refer to transfection of hosts in vitro or in vivo, using nanoparticles of the present invention.

The term “wild-type” (WT), as used herein, refers to the typical, most common or conventional form as it occurs in nature. Thus, the present invention includes methods of gene therapy whereby polynucleotides encoding the desired gene product (such as interferon-gamma) are delivered to a subject, and the polynucleotide is expressed in vivo. The term “gene therapy”, as used herein, includes the transfer of genetic material (e.g., polynucleotides) of interest into a host to treat or prevent a genetic or acquired disease or condition phenotype, or to otherwise express the genetic material such that the encoded product is produced within the host. The genetic material of interest can encode a product (e.g. a protein, polypeptide, peptide, or functional RNA) whose production in vivo is desired. For example, the genetic material of interest can encode a hormone, receptor, enzyme, polypeptide or peptide of therapeutic value. For a review see, in general, the text “Gene Therapy” (Advances in Pharmacology 40, Academic Press, 1997). The genetic material may encode a product normally found within the species of the intended host, or within a different species. For example, if the polynucleotide encodes interferon-gamma, the cytokine may be human interferon-gamma, or that of another mammal, for example, regardless of the intended host. Preferably, the polynucleotide encodes a product that is normally found in the species of the intended host. Alternatively, the genetic material may encode a novel product.

Two basic approaches to gene therapy have evolved: (1) ex vivo and (2) in vivo gene therapy. The methods of the subject invention encompass either or both. In ex vivo gene therapy, host cells are removed from a patient and, while being cultured, are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient.

In in vivo gene therapy, target host cells are not removed from the subject, rather the gene to be transferred is introduced into the cells of the recipient organism in situ, that is within the recipient. Alternatively, if the host gene is defective, the gene is repaired in situ.

The particle of the present invention is capable of delivery/transfer of heterologous nucleic acid sequences into a prokaryotic or eukaryotic host cell in vitro or in vivo. The particle may include elements to control targeting, expression and transcription of the nucleic acid sequence in a cell selective manner as is known in the art. It should be noted that often the 5′UTR and/or 3′UTR of the gene may be replaced by the 5′UTR and/or 3′UTR of other expression vehicles.

Optionally, the particles of the invention may have biologically active agents other than polynucleotides as a component of the complex (either instead of, or in addition to, polynucleotides). Such biologically active agents include, but are not limited to, substances such as proteins, polypeptides, antibodies, antibody fragments, lipids, carbohydrates, and chemical compounds such as pharmaceuticals. The substances can be therapeutic agents, diagnostic materials, and/or research reagents.

The present invention includes pharmaceutical compositions comprising an effective amount of particles of the invention and a pharmaceutically acceptable carrier. The pharmaceutical compositions of the subject invention can be formulated according to known methods for preparing pharmaceutically useful compositions. As used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils.

The pharmaceutically acceptable carrier can be one adapted for a particular route of administration. For example, if the particles of the present invention are intended to be administered to the respiratory epithelium, a carrier appropriate for oral or intranasal administration can be used.

Formulations are described in a number of sources which are well known and readily available to those skilled in the art. For example, Remington's Pharmaceutical Sciences (Martin E. W., 1995, Easton Pa., Mack Publishing Company, 19^{th} ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The terms “comprising”, “consisting of” and “consisting essentially of” are defined according to their standard meaning. The terms may be substituted for one another throughout the instant application in order to attach the specific meaning associated with each term.

As used in this specification and the appended claims, the singular forms “a”, “an”, and “the” include plural reference unless the context clearly dictates otherwise. Thus, for example, a reference to “a particle” includes more than one such particle, a reference to “a polynucleotide” includes more than one such polynucleotide, a reference to “a polypeptide” includes more than one such polypeptide, a reference to “a host cell” includes more than one such host cell, and the like.

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al. (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659; and 5,272,057; and incorporated herein by reference. Polymerase chain reaction (PCR) was carried out generally as in PCR Protocols: A Guide To Methods And Applications, Academic Press, San Diego, Calif. (1990). In situ (In-cell) PCR in combination with Flow Cytometry can be used for detection of cells containing specific DNA and MRNA sequences (Testoni et al., Blood, 1996, 87:3822.)

All patents, patent applications, provisional applications, and publications referred to or cited herein, whether supra or infra, are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### Preparation of Chlipids

A. Materials and Methods

The plasmid pEGFP was propagated in E.coli DH5α cells. Large-scale plasmid DNA was prepared using a QIAGEN kit (QIAGEN, Chatsworth, Calif.), following the manufacturer's specifications. This produced sufficiently pure DNA.

Chlipids were prepared by mixing binary complexes of LIPOFECTIN and DNA with chitosan using procedures previously described for LIPOFECTIN and DNA alone (Miyasaki S. et al., Biol. Pharm. Bull., 1994, 17(5):745-747). This procedure is highly reproducible and nanoparticle yields were similar to those of the chitosan-DNA complexes.

Chitosan (0.01% in Na-acetic acid pH 5.4) was prepared as described previously and 100 μl of chitosan solution was incubated at 55° C. for 10 minutes. Twenty-five μg of DNA was resuspended in 100 μl of sodium sulfate at 55° C. for 10 minutes and then added with 25 μl of lipofectin. The chitosan and lipofectin-DNA solution was mixed and then vortexed for 20 seconds. The preparation was examined under a light microscope. After incubation, nanoparticle-DNA complexes were subjected to analysis by electrophoresis on an agarose gel (1%, ethidium bromide included for visualization) for 90 minutes at 90 V. Images were taken using a UV transilluminator and a GELDOC 2000 gel documentation system (BIORAD). Band integration and background correction was performed using Molecular Analyst Version 1.1 software (BIORAD). To determine optimal serum concentration, A-549 cells were seeded (0.4×10E5 cells/well) in 8-chambered slide microwells and grown in the medium with different serum levels and transfected after 24 h with (0.05%) chitosan complexed with 1 ug DNA and 5 ul of lipofectamin (INVITROGEN, CA.). After 48 hrs the % GFP positive cells were quantified by enumeration of total number cells determined staining with DAPI and GFP positive cells as visualized under a fluorescent microscope. Also, A-549 cells were transfected with pGFP (1 ug) and different lipid conc. With or without chitosan and the percentage of GFP positive cells was quantified as described above.

To determine the nature and size of the chlipids, the particles were analyzed by transmission electron microscope (TEM) for further characterization. The particles were applied for 2 minutes to the carbon surface of 400 mesh copper electron microscope grids covered with Formvar and carbon films and then inverted over 100 μl water droplets on paraffin for 1 minute. The samples were stained with uranyl acetate (0.04% in methanol) for 2 minutes, and then the grids were dipped in ethanol, blotted, and air-dried. Grids were examined using a PHILIPS CM-10 transmission electron microscope. The film plates were exposed to the image at a magnification of 7,700 to 44,000-fold.

B. Results

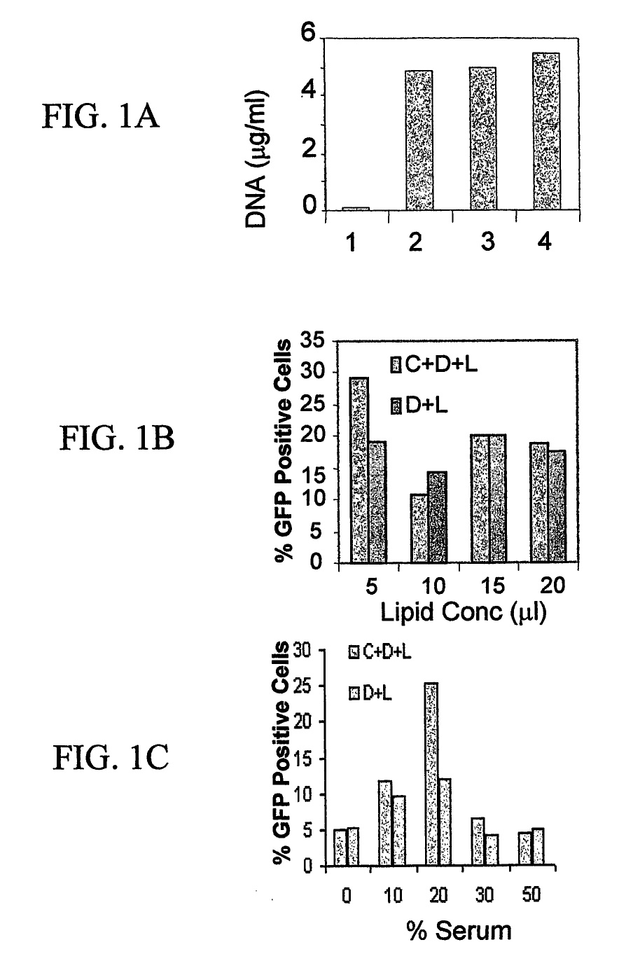

To characterize chlipids prepared using chitosan and lipofectin, the particles complexed with DNA were observed in the gel (data not shown). The complex formation of chitosan with lipid and DNA reproducibly encapsulated a minimum of 50 % of available DNA, irrespective of the concentration of chitosan used. The analysis of gene expression levels shows that both serum concentrations and lipid concentration influence the percentage transfection efficiency. Twenty percent serum and 1:5 ratio of DNA:lipid was found to give the highest GFP gene expression in vitro (FIGS. 1A-1C).

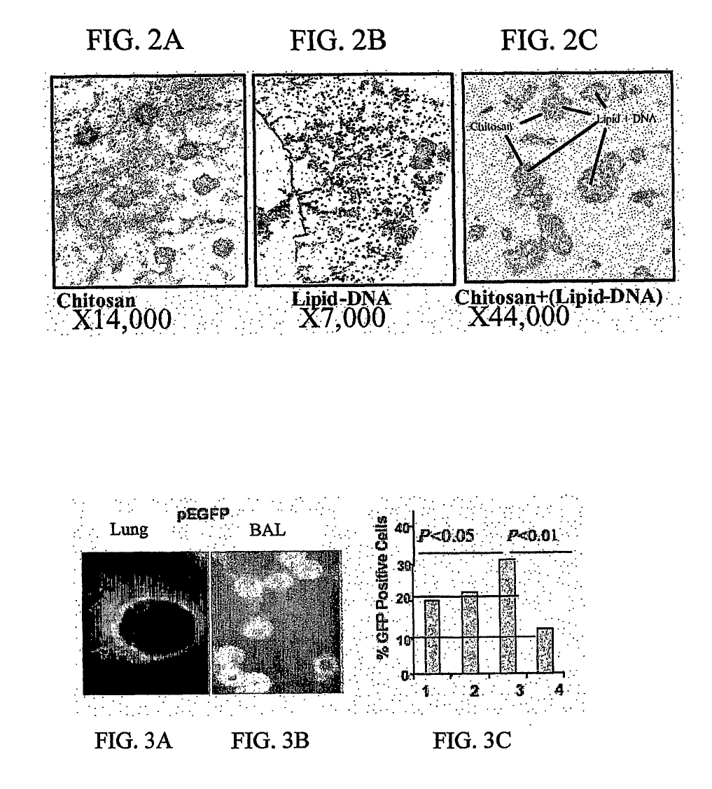

To determine the nature and size of the particles, chlipids were subjected to analysis by TEM. FIGS. 2A-2C show electron micrographs of chitosan at 14,000×, lipid-DNA at 7,000×, and chitosan+(lipid-DNA) at 44,000×, respectively. The shapes of the chlipids were changed slightly but were largely spherical and similar to that of the chitosan particles. Lipid-DNA complexes were visible as electron dense particles and they were impregnated with each chitosan particle. The diameters of both chitosan alone and chitosan complexed with lipids were determined. The sizes of the chitosan-DNA complexes were in the range of 1 μm (1114±114). The sizes of the lipid-DNA binary complexes were in the range of 186±63. However, the sizes of the chitosan-lipid-DNA multiplexes were in the nanometer range, 440±97.

### EXAMPLE 2

### Chlipids Administered Intranasally Transfect Epithelial Cells in the Mouse Lung

A. Materials and Methods

Female 8 week-old BALB/c mice from Jackson Laboratory (Bar Harbor, Me.) maintained in pathogen-free conditions. Mice were intranasally (i.n.) administered under light anesthesia with 100 μl of Chlipids+10 μg of plasmid DNA encoding enhanced green fluorescence protein (EGFP) over a period of three days. Mice were sacrificed on day four and their lungs were lavaged with 1 ml of PBS introduced through the trachea. The BAL fluid was centrifuged for 10 minutes at 300 ×g. Cells were then rinsed with PBS and re-suspended. Mice were given PBS as control.

B. Results

To identify the cells in the lung that are transfected, ovalbumin-sensitized 8 week-old BALB/c mice (n=2 for each group) were given intranasally (30 μg./mouse) using either chlipid complexed with pEGFP or pVAX. Mice were given naked DNA as a control. The results of a representative experiment are shown FIG. 3A. The green fluorescence seen in the lung section suggests that the epithelial cells are predominantly transfected by chlipids. This result is not different from chitosan alone (not shown). However, under low magnification there is sporadic green fluorescence throughout the lung, suggesting that chlipids also transfect lung parenchyma in the distal lung. No green fluorescence was observed in sections from control mice.

### EXAMPLE 3

### Chlipids Induce Enhanced Gene Transfection and Expression in the Lung

A. Materials and Methods

To determine whether chlipid nanoparticles enhance the transfection efficiency in the target lung epithelial cells and monocytes, groups of BALB/c mice were administered intranasally (i.n.) under light anesthesia with 25μg of total pEGFP DNA/mouse complexed with either chitosan alone, lipofectin alone or chlipids prepared as described in Example 1. Control mice received the same amount of DNA in saline PBS. Twenty-four hours after, mice were sacrificed.

A parallel group of mice were subjected to bronchoalveolar lavage. The BAL fluid was centrifuged for 10 minutes at 300×g. Cells were then rinsed with PBS and resuspended. Flow cytometry experiments were conducted to determine the EGFP transfection levels in BAL cells. Aliquots of the cell suspension were applied to slides using a cytospin apparatus (SHANDON SOUTHERN) and the EGFP-positive cells were observed under a fluorescent microscope. A student's t test was performed to determine whether the means differed with level of significance set at p<0.05.

B. Results

Cytospun BAL cells were visualized under a fluorescent microscope to identify GFP expressing cells (FIG. 3B). Only a small subset of cells was found to exhibit green fluorescence. The percent EGFP-positive cells for different groups were plotted (FIG. 3C). The chlipids induced a 30% transfection rate in the lung cells, which was significantly different from that of naked DNA (p<0.01) and from chitosan and lipofectin (p<0.05). These results demonstrate that chlipids provide increased efficiency of transfection and gene expression in the lung cells in vivo.

### EXAMPLE 4

### Chlipids Induce Decreased IL-6 Levels Compared to Chitosan-pVAX Complexes

A. Materials and Methods

BAL fluid pooled from 4 mice of Example 3 was analyzed for IL-6 content using ELISA from an R & D Systems Kit (Minneapolis, Minn.).

B. Results

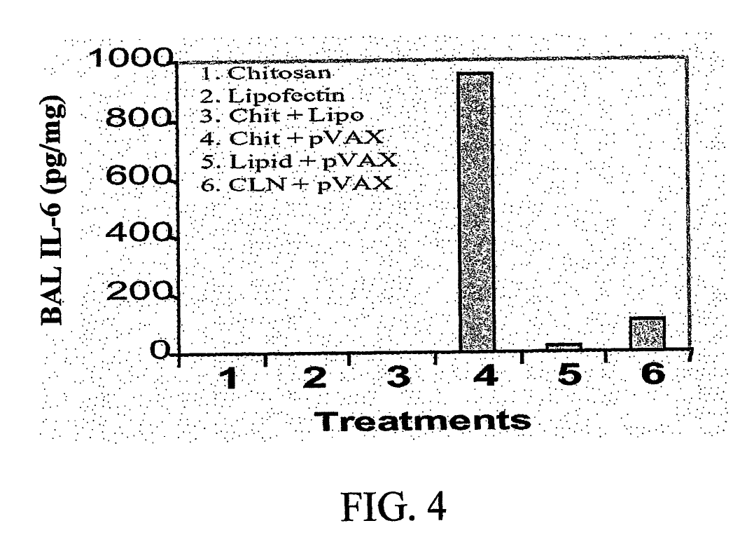

Chitosan-DNA complexes induce production of IL-6, a marker of acute inflammation in the lung. To determine whether chlipids alter the level of IL-6 production, mice were given (i.n.) complexes of chitosan, lipofectin, or chlipid with the vector plasmid pVAX and IL-6 production was examined after 4 hours. Quantification of IL-6 in BAL fluid showed that chlipids induced significantly decreased IL-6 levels compared to chitosan-pVAX complexes, as shown in FIG. 4.

A major finding of the experiments described herein is that chlipids of the present invention have a smaller size compared to chitosan, as evident from TEM analysis. These estimations are in agreement with a previous report (Miyazaki, S. et al. Biol. Pharm. Bull., 1994, 17:745). Of importance is the reduction in size of chlipids (from 1114 nm to 440 nm). This may be due to compaction of chitosan during multiplexing. The structure of the lipid-DNA complex resembles a 2D columnar inverted hexagonal structure in which the DNA molecules are surrounded by a lipid monolayer with the DNA-lipid inverted cylindrical micelles arranged in a hexagonal lattice. It is likely that the chitosan-lipid DNA multiplex forms when DNA simultaneously coacervates with both the cationic lipid and chitosan.

Another significant result is that chlipids induced a significant increase in the transfection of lung cells. These results show that chitosan and lipid exhibit similar transfection efficiencies in vivo, in contrast to in vitro results, where cationic lipids exhibit significantly increased transfection efficiency compared to chitosan. The reason for the increased efficiency of chlipids could be due to a combination of chitosan's biomuco-adhesive ability and the superior transfection efficiency of cationic lipids. These lipids tend to bind to the cells via their net positive charge, with adhesion facilitated by the interaction between positively charged particles and the negatively charged cell membrane.

In addition, chlipids of the present invention induce significantly less IL-6 compared to that induced by chitosan. IL-6 is a marker of acute inflammation and an important index for the safety of these nanoparticles. Chitosan, although inert, does induce inflammation, as is evident from its ability to induce IL-6. Chitosan was previously shown to stimulate macrophages to produce TNF-α, which was augmented by its interaction with CD14 (Richardson, S. C. and Kolbe, H. V. Int. J. Pharm., 1999, 178:231). It is likely that multiplexing with lipids alters chlipid interaction with innate immune receptors on the cell membrane, resulting in a decrease in IL-6 production. Irrespective of the mechanism involved, the evidence that chlipids produce less IL-6 compared to chitosan suggests that chlipids may be safer in the clinical realm.

### EXAMPLE 5

### Expression of IFN-γ from Chitosan complexed with a pDNA expressing cytokine IFN-gamma (CIN) in Lung

A. Materials and Methods

Female 6 to 8 week-old wild-type and STAT4^{-/-} BALB/c mice from Jackson Laboratory (Bar Harbor, Me.) were maintained in pathogen free conditions at the animal center at the University of South Florida College of Medicine. All procedures were reviewed and approved by the committees on animal research at the University of South Florida College of Medicine and VA Hospital.

IFN-γ cDNA was cloned in the mammalian expression vector pVAX (Invitrogen, San Diego, Calif.), and prepared, as described before (Kumar, M. et al. J Allergy Clin Immunol, 2001, 108:402-408). Ten μg of DNA dissolved in 100 μl of Na₂SO₄ solution and heated for 10 min at 55° C. Chitosan (Vanson, Redmond, Wash.) was dissolved in 25 mM Na acetate, ph 5.4 to final concentration of 0.02% in 100 μl volume and heated for 10 min at 55° C. Following incubation, chitosan and DNA were mixed and vortexed vigorously for 20-30 sec and stored at room temperature until use.

B. Results

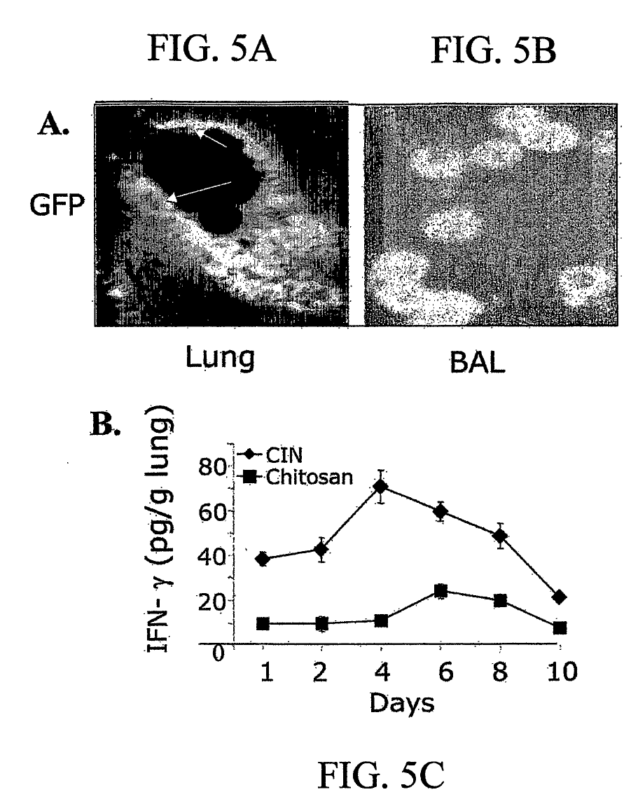

To determine the type of lung cells expressing the chitosan-delivered gene, plasmid DNA (pDNA) expressing a green-fluorescent protein (GFP) was administered intranasally (i.n.) to mice. One day later, the lung sections from one group of mice and the BAL fluid from a parallel group of mice were examined for GFP expression by fluorescence microscopy. Lung sections showed that the GFP was expressed principally by epithelial cells, while in BAL fluid, monocytic cells expressed GFP (FIGS. 5A and 5B, respectively). To examine the time course of gene expression, CIN or chitosan alone was administered to groups of mice (n=3) and the level of expressed IFN-γ was determined by analysis of lung homogenates from each group 1, 2, 4, 6, 8 or 10 days after CIN administration. The results show that CIN rapidly induces IFN-γ expression and the level continues to increase until day 4. However, by day 10 the IFN-γ level in the lung is back to the base level, as shown in FIG. 5C. These results show that intranasal CIN administration promotes IFN-γ production in the lung and that expression primarily occurs in lung epithelial cells and monocytes.

### EXAMPLE 6

### Prophylactic Administration of CIN Attenuates-Allergen-induced AHR and Inflammation

A. Materials and Methods

Prevention of Airway hyperresponsiveness (AHR). Mice were given intranasally 25 μg of chitosan-IFN-γ nanoparticles per mouse daily days 1 through 3. On day 4, mice were sensitized by i.p. injection of 50 μg of OVA adsorbed to 2 mg of aluminum potassium sulfate (alum). On day 19, mice were challenged intranasally with OVA (50 μg per mouse). One day following the last challenge, on day 22, AHR to increasing concentrations of methacholine was measured in conscious mice. On day 23, mice were bled and then sacrificed. Bronchial lymph nodes and lungs were removed and single-cell suspensions of bronchial lymph node cells were prepared and cultured in vitro either in the presence of 100 μg/ml OVA or medium alone.

Measurement of AHR. Airway hyperresponsiveness to inhaled methacholine was measured using the whole body plethysmograph (BUXCO, Troy, N.Y.), as described before (Matsuse, H. et al. J Immunol, 2000, 164:6583-6592).

OVA-specific IgE analysis. To determine the titer of OVA-specific IgE, a microtiter plate was coated overnight at 4° C. with 100 μl of OVA (5 mg/ml). Following three washes, nonspecific sites were blocked with PBST (0.5% Tween-20 in PBS). Mouse sera were added to the antigen-coated wells, the plates were incubated, and bound IgE was detected with biotinylated anti-mouse IgE (02112D; Pharmingen, Calif.). Biotin anti-mouse IgE (02122D) reacts specifically with the mouse IgE of the Igh^{a} and Igh^{b} haplotypes and does not react with other IgG isotypes. Diluted streptavidin-peroxidase conjugate was added, the bound enzyme detected using TMB, and the absorbance read at 450 nm.

Statistical analysis. Values for all measurements are expressed as means±SDs. Pairs of groups were compared through use of Student's t tests. Differences between groups were considered significant at p<0.05.

B. Results

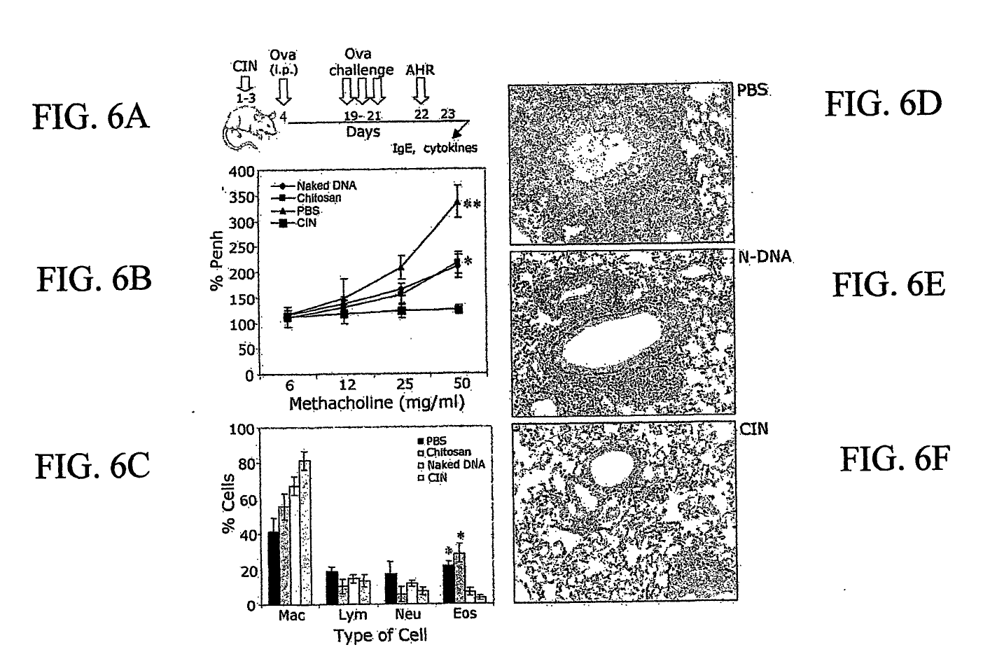

IFN-γ promotes a Th1-like response to allergens. To determine whether 10 prophylactic administration of CIN attenuates sensitization to allergens, mice were first given CIN therapy and then sensitized and challenged with OVA, as shown in the schematic of FIG. 6A. The effect of CIN therapy on airway hyperreactivity was measured by whole body plethysmography. CIN-treated mice showed a significantly (p<0.01) attenuated AHR (% Penh) compared to non-treated mice or mice given the IFN-γ plasmid alone as naked DNA (FIG. 6B). Furthermore, analysis of the cellular composition of the BAL fluid from CIN-treated mice showed a doubling of monocytes, while in the lungs there were significant reductions in the numbers of eosinophils (FIG. 6C). Histological examination of lung sections (FIGS. 6D, 6E, and 6F) revealed that CIN-treated mice exhibited a significant decrease in epithelial denudation, mucus cell metaplasia, and cellular infiltration compared to non-treated mice or mice given naked IFN-γ plasmid.

### EXAMPLE 7

### Prophylactic Administration of CIN Attenuates Cytokine Production to Allergens

A. Materials and Methods

Bronchial lymph node culture and assay for cytokines. Single-cell suspensions of bronchial lymph nodes (3×10⁵ cells/well of a 24-well plate) were re-stimulated in vitro in the presence or absence of 100 μg/ml OVA. Supernatants were collected after 48 h for cytokine ELISA. ELISAs for IL-4, IL-5, and IFN-γ were done using kits from R & D Systems (Minneapolis, Minn.), following the manufacturer's protocols.

B. Results

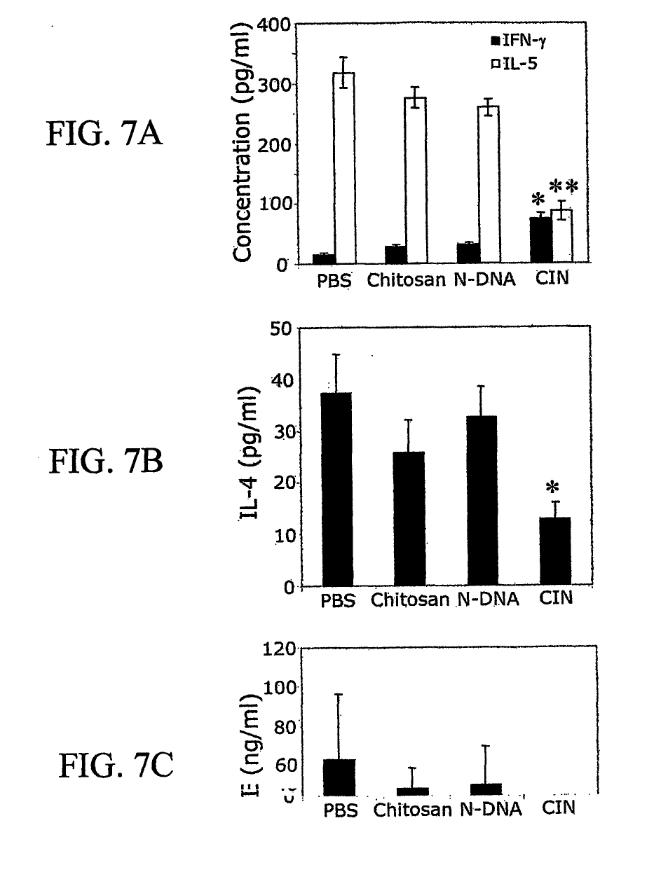

To determine whether the significant reduction in AHR in CIN-treated mice was due to attenuated allergen sensitization, Th2 cytokines were measured in splenocytes from the three groups of mice. The CIN-treated mice showed significant reduction in the amount of IL-5 and IL-4 compared to control mice (FIGS. 7A and 7B, respectively). In contrast, IFN-γ secretion was significantly higher in CIN treated mice compared to control mice (FIG. 7A). CIN-treated mice also showed a significant reduction in IgE antibody levels compared to the control group (FIG. 7C). These results indicate that CIN prophylaxis results in the attenuation of allergen sensitization.

### EXAMPLE 8

### Therapeutic Administration of CIN Reverses Established Allergen-induced AHR

A. Materials and Methods

Reversal of established AHR. Mice were sensitized i.p. with 50 μg OVA on day 1 followed by intranasal challenge with 50 μg of OVA on day 14. On day 21-23, mice were given intranasally 25 μg of chitosan-IFN-γ nanoparticles per mouse. Mice were further challenged i.n. with OVA (50 μg/mouse) on days 27 through 29 and AHR was measured on day 30. Mice were bled and sacrificed on day 31, as described for the earlier protocol.

B. Results

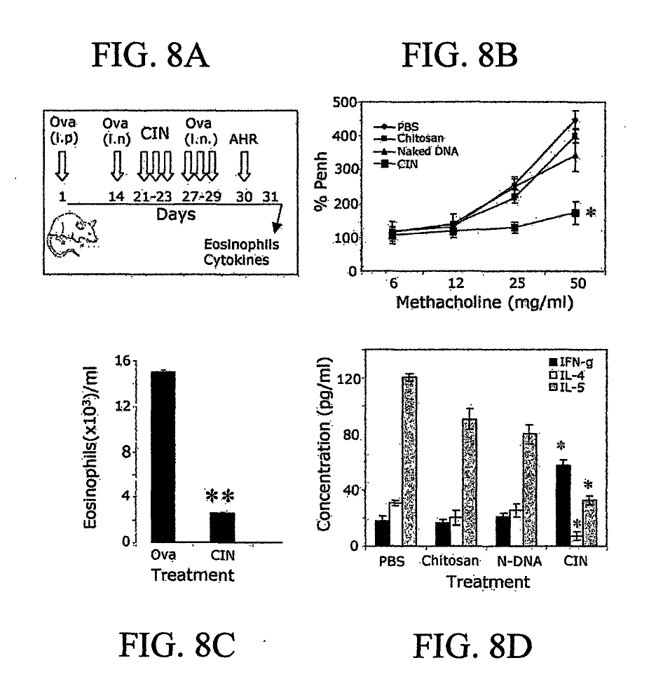

Intranasal Ad-IFN-γ is capable of reversing established AHR (Behera, A. K. et al. J Biol Chem, 2002, 277:25601-8). To determine whether therapeutic administration of CIN can attenuate established asthma, mice were first sensitized and challenged with OVA and then given CIN therapy, as shown in the protocol depicted in FIG. 8A. Airway hyperreactivity (% Penh) was measured by whole body plethysmography (FIG. 8B) and CIN-treated mice again had lower AHR than those mice given chitosan alone or IFN-γ plasmid alone. The results show a complete reversal to the basal level of AHR in the group of mice that were treated with CIN. Upon staining the lung sections with an antibody against Muc5a, a marker that is specific for mucus-producing cells, the number of eosinophils in the BAL fluid showed a significant reduction in the CIN-treated mice (FIG. 8C) compared with the untreated control group. Furthermore, analysis of cytokine secretion from splenocytes showed that there was an increase in IFN-γ production and a decrease in IL-4 and IL-5 production in the CIN-treated mice compared to the controls (FIG. 8D).

### EXAMPLE 9

### Therapeutic Administration of CIN Reverses Established Allergen-induced Inflammation by Apoptosis of Inflammatory Cells

A. Materials and Methods

Lung histology and apoptosis assay. Mice were sacrificed within 24 hours after the last challenge, and lung sections were paraffin embedded. Lung inflammation was assessed after the sections were stained with hematoxylin and eosin. Unstained sections were examined for apoptosis by the TUNEL (terminal deoxynucleotidyl transferase dUTP nick end-labeling) assay method according to manufacturer's instructions (DEADEND Fluorometric TUNEL Assay, Promega, Madison, Wis.), as described (Hellermann, G. R. et al. Resp. Res., 2002, 3:22-30). Briefly, lung sections were dewaxed in xylene, rehydrated, and fixed with 4% paraformaldehyde for 15 min. Sections were then washed three times in PBS, permeabilized 15 min with 0.1% Triton X-100, and incubated one hour at 37° C. with the TUNEL reagent. The reaction was terminated by rinsing slides once with 2×SSC and three times in PBS. The lung sections were observed microscopically and green fluorescence photographed using a Nikon TE300 fluorescence microscope with a digital camera.

B. Results

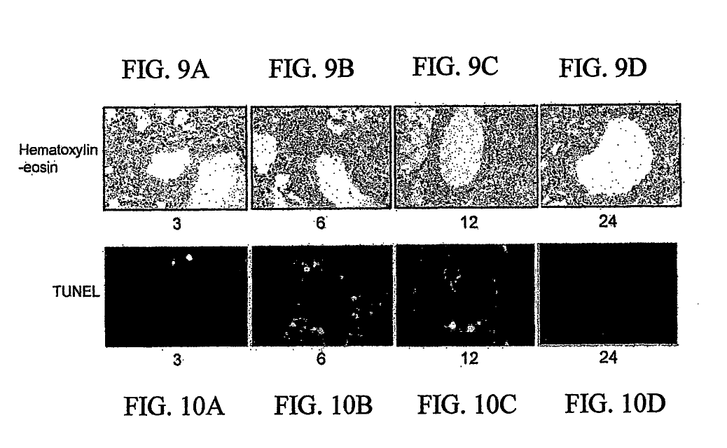

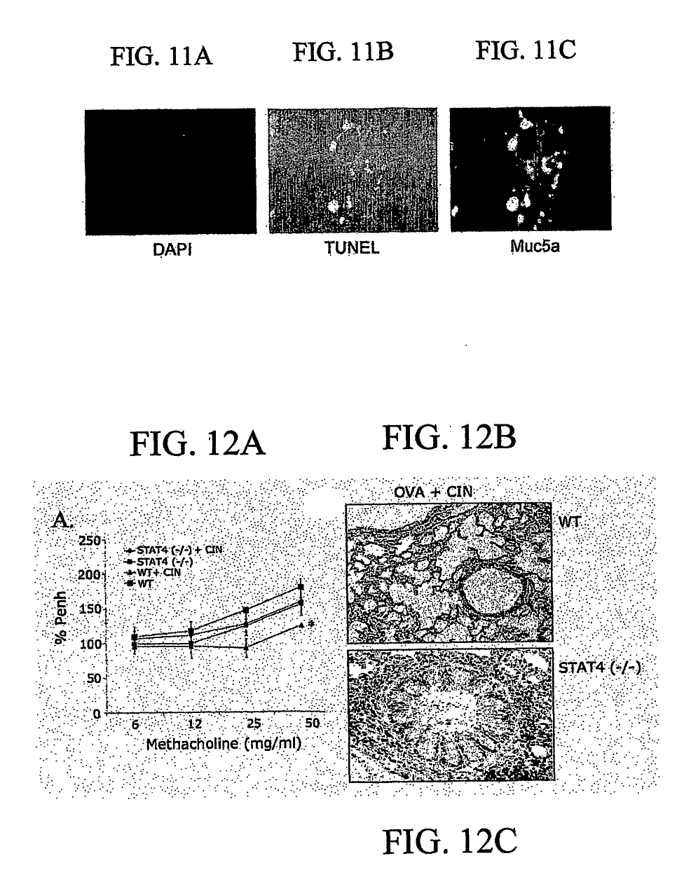

To determine whether CIN therapy decreases established pulmonary inflammation, lungs from OVA-sensitized and OVA-challenged mice were examined 3, 6, 12, and 24 hours after CIN administration. Histopathologic analysis of the bronchial epithelium showed that goblet cell hyperplasia began to attenuate after 6 hours of CIN administration (FIGS. 9A-9D). Staining of lung sections for apoptosis (TUNEL assay) showed a significant number of TUNEL-positive cells at 6 hours and 12 hours after CIN administration, which was back to normal by 24 hours (FIGS. 9A-9D). In FIGS. 11A-11C, the cells undergoing apoptosis (TUNEL) were identified as goblet cells by staining the lung sections with mucus cell-specific marker, Muc5a. These results indicate that CIN reverses epithelial inflammation rapidly within hours.

### EXAMPLE 10

### CIN Therapy Involves the STAT4 Signaling Pathway

Ad-IFN-γ gene transfer, which produces significant amounts of IFN-γ in the lung, has been shown to involve the IL-12/ STAT4 signaling pathway (Hellermann, G. R. et al., Resp. Res. 2002, 3:22-30). To determine whether CIN also uses a STAT4 pathway, CIN therapy was tested on STAT4-deficient mice (STAT4^{-/-}). Wild-type mice showed the expected reduction in % Penh with CIN treatment while the STAT4-deficient mice had no significant change in AHR after CIN treatment (FIG. 12A). Lung histopathology analysis of wild-type and STAT4^{-/-} mice treated with CIN showed that CIN did not protect the lungs of STAT4^{-/-} mice against inflammation (FIGS. 12B and 12C). These results suggest that STAT4 signaling is significant in the effectiveness of CIN therapy.

The role of IFN-γ in modulating allergen-induced asthma has been described by many investigators (Kumar, M. et al. Human Gene Therapy, 2002, 13:1415-25; Matsuse, H. et al. J Immunol, 2000, 164:6583-6592; Behera, A. K. et al. J Biol Chem, 2002, 277:25601-8). Using mouse models, a variety of approaches have been tried, ranging from i.p. administration of recombinant IFN-γ to adenovirus-mediated gene transfer (Flaishon, L. et al. J Immunol, 2002, 168:3707-11; Yoshida, M. et al. Am J Respir Crit Care Med, 2002, 166:451-6). However, none of these approaches may be suitable for utilizing IFN-γ therapy in humans. In the experiments set forth herein, a non-viral intranasal gene transfer strategy is described using a human-friendly gene carrier, chitosan. The results in a mouse model of allergic asthma demonstrate that CIN therapy is potentially an effective prophylactic and therapeutic treatment for asthma. Evidence is also presented that, analogous to other anti-inflammatory therapies, the immune modulation of CIN therapy is STAT4 dependent.

Although chitosan has been previously administered intranasally, the pattern of gene expression mediated by chitosan nanoparticles has not been studied. The results of this study show that the bronchial epithelium is the major target of chitosan nanoparticles. In addition to epithelial cells, macrophages appeared to also take up chitosan nanoparticles. Both of these cell types play an important role in asthma and in immunomodulation (Tang, C. et al. J Immunol., 2001, 166:1471-81). A major drawback of the adenovirus-mediated gene transfer is that entry into bronchial epithelial cells requires the CAR receptor, which is expressed on the basolateral, but not the apical, surface of epithelial cells. Mucus may also interfere with adenoviral gene transfer, whereas chitosan has been shown to have muco-adhesive properties (Filipovic-Grcic, J. et al. J Microencapsul, 2001, 18:3-12). The role of monocytes is important, as monocytes are activated in response to IFN-γ production, which leads to IL-12 production and amplification of the IFN-γ cascade (Hayes, M. P. et al. Blood, 1995, 86:646-50). The time course of IFN-γ expression through delivery of CIN is also distinct from that of adenoviral-mediated IFN-γ expression in that the amount of IFN-γ expression is lower, but the duration of IFN-γ production is prolonged.

A significant finding was that treatment with CIN reversed the course of asthma, as is evident from the normalization of AHR and the return to normal lung morphology from the hyper-inflammatory condition induced by OVA sensitization and challenge. This result is consistent with our previous observations and those of others. Furthermore, the reduction in eosinophilia was greater with CIN therapy than with Ad-IFN treatment. A novel finding is that chitosan IFN-γ works within 3-6 h after intranasal administration, as mucus cell metaplasia was reduced as early as 6 h after treatment. This reduction is seen despite the fact that CIN therapy produces about 10-fold less IFN-γ than Ad-IFN-γ treatment. The effective transfection of lung epithelial cells by CIN may account for this increased effectiveness.

In conclusion, intranasal CIN treatment may be useful for both prophylaxis and treatment of asthma.

It should be understood that the examples and embodiments described herein are for illustrative purposes only and that various modifications or changes in light thereof will be suggested to persons skilled in the art and are to be included within the spirit and purview of this application.

1. A particle comprising chitosan, or a derivative thereof; and a polynucleotide.

2. The nanoparticle of claim 1, wherein said particle further comprises a lipid, and wherein said particle comprises a complex of said chitosan, said polynucleotide, and said lipid.

3. The particle of claims 1, wherein said polynucleotide encodes a cytokine.

4. The particle of claim 1, wherein said polynucleotide encodes interferon gamma.

5. A composition comprising a particle and a pharmaceutically acceptable carrier, wherein said particle comprises chitosan, or a derivative thereof, and a polynucleotide.

6. The composition of claim 5, wherein said particle further comprises a lipid, and wherein said particle comprises a complex of said chitosan, said polynucleotide, and said lipid.

7. The composition of claim 5, wherein said polynucleotide encodes a cytokine.

8. The composition of claim 5, wherein said polynucleotide encodes interferon gamma.

9. (canceled)

10. A method for delivery and expression of a polynucleotide within a host, said method comprising administering a particle to the host, wherein the particle comprises chitosan, or a derivative thereof, and a polynucleotide.

11. The method of claim 10, wherein the particle further comprises a lipid, and wherein the particle is a complex of the chitosan, polynucleotide, and lipid.

12. The method of claim 10, wherein the polynucleotide encodes a cytokine.

13. The method of claim 10, wherein the polynucleotide encodes interferon gamma.

14-15. (canceled)

16. The method of claim 10, wherein the particle is administered within a composition comprising a pharmaceutically acceptable carrier.

17. A method for enhancing interferon-gamma expression to regulate the production of cytokines secreted by T-helper type 2 (Th2) cells, said method comprising administering an effective amount of a particle to a subject, wherein the particle comprises chitosan, or a derivative thereof, and a polynucleotide encoding interferon-gamma.

18. The method of claim 17, wherein the subject is human.

19. The method of claim 17, wherein the subject is suffering from asthma.

20. The method of claim 17, wherein the particle is administered to the respiratory tract of the subject.

21. A method for producing a particle comprising a complex of chitosan, or a derivative thereof and a polynucleotide, said method comprising mixing the polynucleotide and the chitosan or chitosan derivative, to form the particle.

22. The method of claim 21, wherein said method further comprises nixing a lipid with polynucleotide and the chitosan or chitosan derivative, wherein the particle comprises a complex of polynucleotide, chitosan or chitosan derivative, and the lipid.

23. (canceled)
